(19) United States
(12) Patent Application Publication
(10) Pub. No.: US 2004/0064168 A1
(43) Pub. Date: Apr. 1, 2004

(54) Thermal pack apparatus

(76) Inventor: Clement G. Eischen SR., Gresham, OR (US)

Correspondence Address: KOLISCH HARTWELL, P.C., 520 S.W. YAMHILL STREET, SUITE 200, PORTLAND, OR, 97204, US

(21) Appl. No.: 10/666,877

(22) Filed: Sep. 18, 2003

Related U.S. Application Data
(60) Provisional application No. 60/414,094, filed on Sep. 26, 2002.

(51) Int. Cl. A61F 7/00

(52) U.S. Cl. 607/96

(57) ABSTRACT

A thermal pack apparatus including a shell at least partially filled with a thermal material. The shell typically includes an outer perimeter, opposed sides of the outer perimeter each having a respective notch formed therein, the notched being sized to enable an upper portion of the shell to be folded to form a first end surrounding a limb on a first side of a joint, and a lower portion of the shell to be folded to form a second end surrounding the limb on a second side of the joint, the shell further including a central portion configured to act as a hinge to enable flexing of the first end relative to the second end, to thereby accommodate movement of the joint.

### CROSS-REFERENCE TO RELATED APPLICATIONS

This application claims the benefit of U.S. Provisional Patent Application No. 60/414,094, titled “Thermal Pack Apparatus,” filed Sep. 26, 2002, the disclosure of which is incorporated herein by reference in its entirety for all purposes.

### TECHNICAL FIELD

The present invention relates generally to thermal packs used to heat or cool a portion of the body.

### BACKGROUND OF THE INVENTION

In athletics and other strenuous activities people often suffer injuries that require treatment by application of heat and cold packs, referred to herein collectively as “thermal packs.” One problem with prior thermal packs is that they are difficult to place around areas of the body that bend, such as jointed limbs.

### SUMMARY OF THE INVENTION

A thermal pack apparatus including a shell at least partially filled with a thermal material is provided. The shell typically includes an outer perimeter, opposed sides of the outer perimeter each having a respective notch formed therein, the notched being sized to enable an upper portion of the shell to be folded to form a first end surrounding a limb on a first side of a joint, and a lower portion of the shell to be folded to form a second end surrounding the limb on a second side of the joint, the shell further including a central portion configured to act as a hinge to enable flexing of the first end relative to the second end, to thereby accommodate movement of the joint.

The advantages of the present invention will be understood more readily after a consideration of the drawings and the Detailed Description of the Preferred Embodiments.

### BRIEF DESCRIPTION OF THE DRAWINGS

FIG. 1 is top view of a thermal pack apparatus according to one embodiment of the present invention, for use on an ankle.

FIG. 2 is a cutaway view of the thermal pack apparatus of FIG. 1.

FIG. 3 is a side view of the thermal pack apparatus of FIG. 1, in a folded configuration.

FIG. 4 is a side view of a thermal pack apparatus according to another embodiment of the present invention, having a side notch with a greater Angle A for greater range of motion, thereby allowing increased joint flexibility.

FIG. 5 is a top view of a thermal pack according to another embodiment of the present invention, for use on shoulders and knees.

FIG. 6 is a side view of the thermal pack apparatus of FIG. 1, shown in stalled on an ankle of a user.

### DETAILED DESCRIPTION OF THE PREFERRED EMBODIMENTS

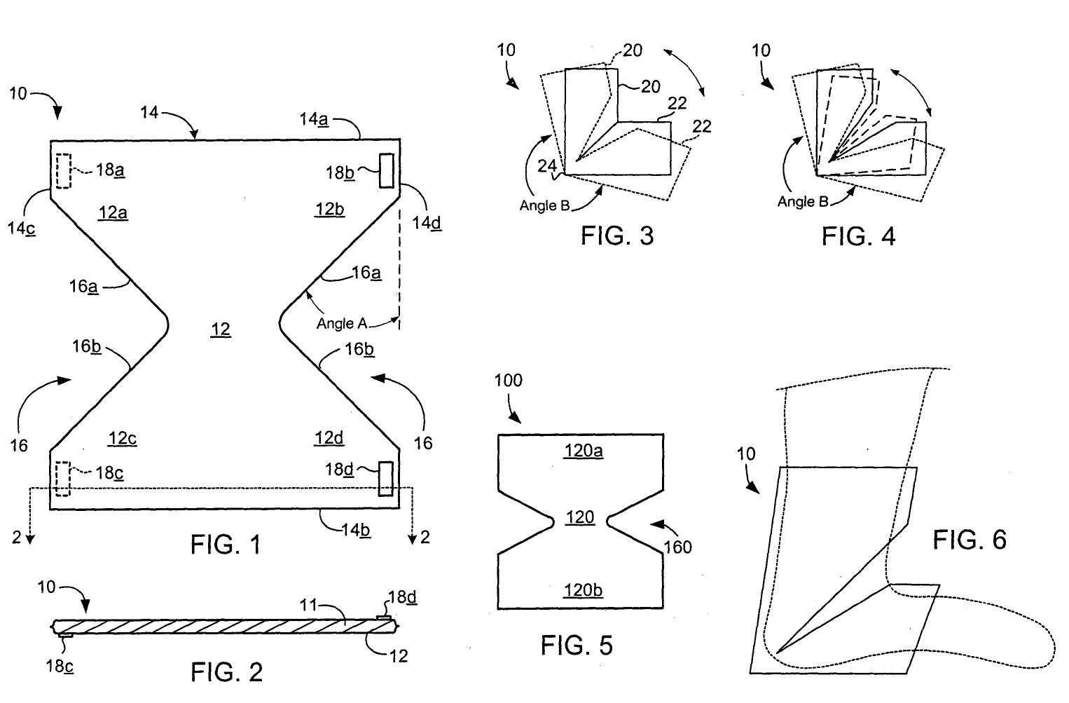

Referring to FIG. 1, a thermal pack apparatus according to one embodiment of the present invention is shown generally at 10. As shown in FIG. 2, thermal pack apparatus 10 typically includes a shell 12 filled with a thermal material 11 such as silica gel having a relatively high thermal mass and low thermal conductivity, thus enabling the material to absorb a large amount of heat energy and slowly transfer that heat energy to a portion of the body, such as a ankle, knee, or shoulder. It will be appreciated that the present thermal pack may be used both in heat and cold applications. Thermal pack apparatus 10 is typically used applied to an ankle, while thermal pack apparatus 100, shown in FIG. 5, is typically applied to a shoulder or knee. Of course, the dimensions of the pack may be adjusted to fit other jointed regions of the body, such as a wrist or neck.

Shell 12 typically includes an outer perimeter 14 having a top 14a, bottom 14b, and right and left sides 14c, 14d, which are typically but not necessarily symmetrical. Each side 14c, 14d typically includes a notch 16 formed by notch sides 16a, 16b that are formed at Angle A relative to the vertical portion of side 14c, 14d. Typically the notch sides 16a, 16b are symmetrical, although asymmetrical sides are also contemplated. While shown as straight, it will also be appreciated that notch sides 16a, 16b may also include curves.

Shell 12 also typically includes tab portions 12a-12d, each including a respective fastener 18a-18d. Fasteners 18a-18d are typically in the form of Velcro fasteners, but also may be straps, buckles, snaps laces, adhesive tape, or virtually any other suitable type of fastener. In use, the upper left portion 12a is folded to attach to upper right portion 12b via fasteners 18a, 18b, and lower left portion 12c is folded to attach to lower right portion 12d via fasteners 18c, 18d, to assume the fastened configuration shown in FIGS. 3-4, and 5.

FIG. 3 shows the thermal pack apparatus of FIG. 1 in a fastened configuration. In the fastened configuration, it will be appreciated that the fastened ends 20, 22, are jointed together by a living hinge 24 formed by the fold in the shell 12. Thus, the ends 20, 22 may be opened and closed and Angle B may change. With this hinging mechanism, the thermal pack apparatus 10 may be placed over a jointed limb, fastened, and accommodate the bending and flexing of the limb.

FIG. 4 shows thermal pack apparatus 10 having a notch 16 formed with a greater Angle A. With a larger notch 16, the ends 20, 22 of the thermal pack apparatus may bend at hinge 24 to form an acute angle (i.e., Angle B>270), as well as an obtuse angle (270>Angle B>180), as shown.

FIG. 6 shows thermal pack apparatus installed on an ankle, in a configuration in which ends 20, 22 are bent at hinge 24 to form an acute angle (i.e. Angle B>270).

FIG. 5 shows a thermal pack apparatus 100 according to another embodiment of the present invention, suitable for placement on a knee or shoulder of a user. Thermal pack apparatus 100 typically includes a shell 120 having a notch 160 formed at a narrower angle, than thermal pack apparatus 10. It will also be appreciated that upper portion 120a and lower portion 120b of apparatus 100 are wider than in apparatus 10.

Although the invention has been disclosed in its preferred forms, the specific embodiments thereof as disclosed and illustrated herein are not to be considered in a limiting sense, because numerous variations are possible. The subject matter of the invention includes all novel and non-obvious combinations and subcombinations of the various elements, features, functions, and/or properties disclosed herein.

I claim:

1. A thermal pack apparatus, comprising:
a shell at least partially filled with a thermal material, the shell including an outer perimeter, opposed sides of the outer perimeter each having a respective notch formed therein, the notched being sized to enable an upper portion of the shell to be folded to form a first end configured to surround a limb on a first side of a joint, and a lower portion of the shell to be folded to form a second end configured to surround the limb on a second side of the joint, the shell further including a central portion configured to act as a hinge to enable flexing of the first end relative to the second end, to thereby accommodate movement of the joint.

2. The thermal pack apparatus of claim 1, wherein the notches are sized to accommodate a knee.

3. The thermal pack apparatus of claim 1, wherein the notches are sized to accommodate an elbow.

4. The thermal pack apparatus of claim 1, wherein the thermal material is a gel.

5. The thermal pack apparatus of claim 4, wherein the thermal material is a silica gel.

6. The thermal pack apparatus of claim 1, wherein the shell further includes a first pair of fasteners positioned respectively on an upper portion of each of the opposed sides, the first pair of fasteners being configured to couple the opposed sides to each other to surround the limb on the first side of the joint.

7. The thermal pack apparatus of claim 6, wherein the shell further includes a second pair of fasteners positioned respectively on an lower portion of each of the opposed sides, the second pair of fasteners being configured to couple the opposed sides to each other to surround the limb on the second side of the joint.

8. The thermal pack apparatus of claim 1, wherein the notch is sized such that the central portion is free to bend to form an acute between the first and second ends of the shell.

9. The thermal pack apparatus of claim 1, wherein the notch is sized such that the central portion is free to bend to form an obtuse angle between the first and second ends of the shell.

10. The thermal pack apparatus of claim 1, wherein the notch is sized such that the central portion is free to bend in one direction to form an obtuse angle, and in an opposite direction to form an acute angle, between the first and second ends of the shell.
